(19) United States
(12) Patent Application Publication
(10) Pub. No.: US 2004/0249409 A1
(43) Pub. Date: Dec. 9, 2004

(54) Reinforced filter membrane

(75) Inventors: Jeff Krolik, Campbell, CA (US); Jackson Demond, Santa Cruz, CA (US); Amr Salahieh, Saratoga, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/457,161

(22) Filed: Jun. 9, 2003

(51) Int. Cl. A61M 29/00

(52) U.S. Cl. 606/200

(57) ABSTRACT

A reinforced filtering device and method of making and using the same. The present invention comprises a filtering device including an elongate shaft and a filter coupled to the shaft. The filter may be reinforced in a number of different ways and by a number of structures including a support fiber.

### FIELD OF THE INVENTION

The present invention pertains to filtering devices. More particularly, the present invention pertains to embolic protection filtering devices having a reinforced filter membrane.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences since the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

### BRIEF SUMMARY

The invention provides design, material, manufacturing method, and use alternatives for intravascular filtering devices. In at least some embodiments, these filtering devices include a shaft having an embolic protection filter coupled thereto, for example adjacent the distal end. The filter may be supported by a support structure. These and other desirable features are described in greater detail below.

### BRIEF DESCRIPTION OF THE DRAWINGS

FIG. 1 is a perspective view of an example filtering device;

FIG. 2 is a side view of the filtering device depicted in FIG. 1, showing the membrane support fibers;

FIG. 3 is a side view of another example filtering device;

FIG. 4 is a side view of another example filtering device;

FIG. 5 is a side view of another example filtering device;

FIG. 6 is a side view of another example filtering device;

FIG. 7 is a side view of another example filtering device;

FIG. 8 is a side view of another example filtering device;

FIG. 9 is a partially cross-sectioned side view of another example filtering device; and

FIG. 10 is a side view of another example filtering device.

### DETAILED DESCRIPTION

The following description should be read with reference to the drawings wherein like reference numerals indicate like elements throughout the several views. The detailed description and drawings illustrate example embodiments of the claimed invention.

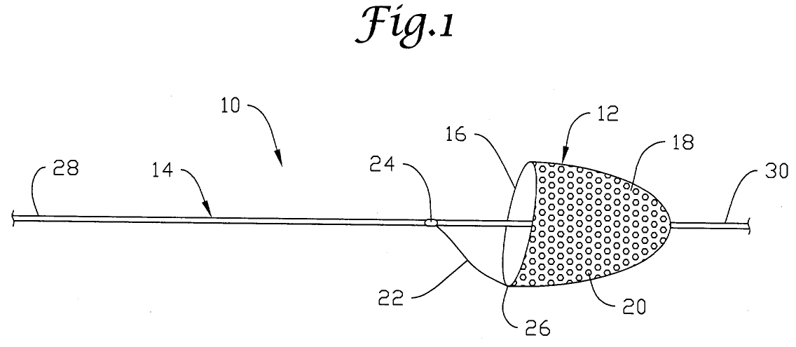

For a number of reasons, it may be desirable to reinforce an embolic protection filter. FIG. 1 is a side view of an example filtering device 10 including a reinforced filter 12 coupled to an elongate shaft 14. Reinforced filter 12 may generally include additional structural support that may help maintain the integrity of filter 12 during an intravascular filtering procedure. The structural support may take on a number of different forms. Some examples of the various forms are discussed in greater detail below in relation to later figures.

In general, filter 12 may be adapted to operate between a first generally collapsed configuration and a second generally expanded configuration for collecting debris in a body lumen. In some embodiments, filter 12 can be delivered to an appropriate intravascular location, for example “downstream” of an intravascular lesion, using an appropriate filter delivery device. Similarly, filter 12 can be removed from the vasculature at the desired time by an appropriate filter retrieval device.

Filter 12 may include a filter frame 16 and a filter membrane or fabric 18 coupled to filter frame 16. Frame 16 may take the form of any one of a number of appropriate shapes and configurations. For example, frame 16 may comprise a generally circular filter mouth or loop, which may defines the primary opening for blood to travel into and be filtered by filter 12. However, essentially any appropriate shape or configuration may be utilized without departing from the spirit of the invention.

Frame 16 may be comprised of any appropriate material. For example, frame 16 may be comprised of a “self-expanding” shape-memory material such as nickel-titanium alloy (to bias filter 12 to be in the second expanded configuration). Alternatively, frame 16 may be comprised of essentially any appropriate metal, metal-alloy, polymer, combinations thereof, and the like including any of the materials described herein. In some embodiments, frame 16 or portions thereof may be doped with, plated with, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, plastic material loaded with a radiopaque filler, and the like. For example, a radiopaque wire disposed about a portion of frame 16.

Filter membrane 18 may be comprised of any appropriate material such as a polymer and may be drilled (for example, formed by known laser techniques) or otherwise include at least one opening 20. Holes or openings 20 can be sized to allow blood flow therethrough but restrict flow of debris or emboli floating in the body lumen or cavity.

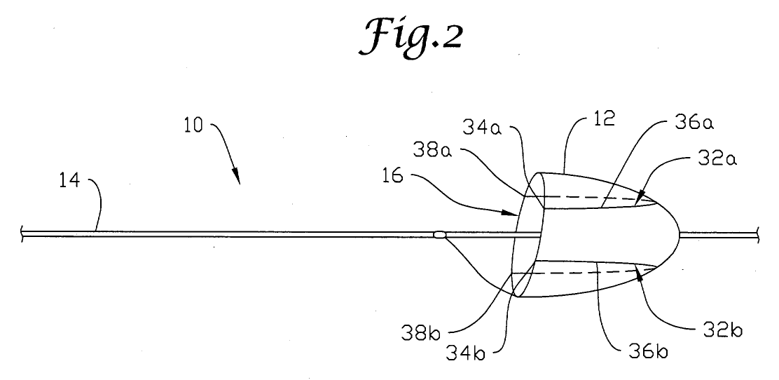

In at least some embodiments, filter membrane 18 extends proximally from frame 16 to define filter 12. Frame 16 may or may not provide any structural support to filter membrane 18. For example, frame 16 may comprise a filter loop and filter member 18 may be coupled to the filter hoop and extend distally, essentially “unsupported” by frame 16. Structural support for membrane 18, therefore, can be derived from a support structure such as support fibers 32a/b, as discussed below in relation to FIG. 2. Additionally, the shape of filter membrane 18 may generally determine the shape of filter 12. For example, filter membrane 18 may define a generally conical, frustoconical, cylindrical, rounded cylindrical, or essentially any other appropriate shape.

One or more struts 22 may extend between frame 16 and shaft 14. In some embodiments, struts 22 can be coupled to shaft 14 by a coupling 24, for example a heat-shrink tube, a crimp fitting, and the like. Alternatively, struts 22 may be coupled to shaft 14 by one or more windings of struts 22 about shaft 14. In some embodiments, struts 22 may comprise an extension or integral part of frame 16. Alternatively, struts 22 and frame 16 may comprise two distinct structures that are attached at an attachment point 26.

Shaft 14 may include a proximal region 28 and a distal region 30, and can be made of any suitable materials including metals, metal alloys, polymers, or the like, or combinations or mixtures thereof. Some examples of suitable metals and metal alloys include stainless steel, such as 304v stainless steel; nickel-titanium alloy, such as nitinol, nickel-chromium alloy, nickel-chromium-iron alloy, cobalt alloy, or the like; or other suitable material. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

The embodiment shown in FIG. 1 illustrates shaft 14 as being a guidewire. However, shaft 14 is not intended to be limited to being only a guidewire. It can be appreciated that shaft 14 may comprise number of different structures including a catheter (e.g., therapeutic, diagnostic, or guide catheter), endoscopic device, laproscopic device, an embolic protection device, or any other suitable device. In some embodiments, shaft 14 may comprise a tubular filter cartridge. According to this embodiment, filtering device 10 can be configured to be slidable over a guidewire or other suitable medical device.

The shaft 14 may include a distal region 30 and a proximal region 28. The entire shaft 14 can be made of the same material, or in some embodiments, can include portions or sections made of different materials. In some embodiments, the material used to construct shaft 14 is chosen to impart varying flexibility and stiffness characteristics to different portions of shaft 14. For example, proximal region 28 and distal region 30 may be formed of different materials, for example materials having different moduli of elasticity, resulting in a difference in flexibility. In some embodiments, the material used to construct proximal region 28 can be relatively stiff for pushability and torqueability, and the material used to construct distal region 30 can be relatively flexible by comparison for better lateral trackability and steerability. For example, proximal region 28 can be formed of straightened 304v stainless steel wire or ribbon, and distal region 30 can be formed of a straightened super elastic or linear elastic alloy, for example a nickel-titanium alloy wire or ribbon.

In embodiments where different portions of shaft 14 are made of different material, the different portions can be connected using any suitable connecting techniques. For example, the different portions of the core wire can be connected using welding, soldering, brazing, adhesive, or the like, or combinations thereof. Additionally, some embodiments can include one or more mechanical connectors or connector assemblies to connect the different portions of the core wire that are made of different materials. The connector may include any structure generally suitable for connecting portions of a guidewire. One example of a suitable structure includes a structure such as a hypotube or a coiled wire which has an inside diameter sized appropriately to receive and connect to the ends of the proximal portion and the distal portion. Some other examples of suitable techniques and structures that can be used to interconnect different shaft sections are disclosed in U.S. patent application Ser. No. 09/972,276, which is incorporated herein by reference.

The length of shaft 14, or the length of individual portions thereof, are typically dictated by the length and flexibility characteristics desired in the final form of device 10. In some example embodiments, proximal portion 20 may have a length in the range of about 20 to about 300 centimeters and distal portion 18 may have a length in the range of about 3 to about 50 centimeters. It can be appreciated that alterations in the length of shaft 14 or portions thereof can be made without departing from the spirit of the invention. For example, in embodiments where shaft 14 is a filter cartridge tube, the length of shaft 14 or portions thereof may be about 0.1 to 20 centimeters or more.

In addition, shaft 14 can have a solid cross-section as shown, but in some embodiments, can have a hollow cross-section. For example, shaft 14 may comprise a tubular catheter or filter cartridge. In yet other embodiments, shaft 14 can include a combination of areas having solid cross-sections and hollow cross sections. Moreover, shaft 14, or portions thereof, can be made of rounded wire, flattened ribbon, or other such structures having various cross-sectional geometries. The cross sectional geometries along the length of the shaft can also be constant or can vary. Additionally, shaft 14 may also include one or more tapered region.

As stated above, filter 12 may include some form of structural reinforcement. For example, filter 12 may include one or more membrane support fibers 32a/b as shown in FIG. 2. Support fibers 32a/b may include a first end 34a/b, a body region 36a/b, and a second end 38a/b. In some embodiments, first ends 34a/b and/or second ends 38a/b are coupled to frame 16. It can be appreciated, however, that ends 34a/b and/or 38a/b can be disposed at essentially any appropriate location. For example, some embodiments of device 10 include first end 34a/b coupled to frame 16 and second ends 38a/b coupled to filter membrane 18 or other suitable locations.

In general, support fibers 32a/b are configured to provide structural support to filter 12. Accordingly, fibers 32a/b may be comprised of a material appropriate for providing sufficient support. For example, fibers 32a/b may be comprised of a metal, polymer, metal-polymer composite, and the like including any of the materials disclosed herein. Alternatively, fibers 32a/b may be comprised of any suitable material, including the same materials as frame 16 and/or filter membrane 18.

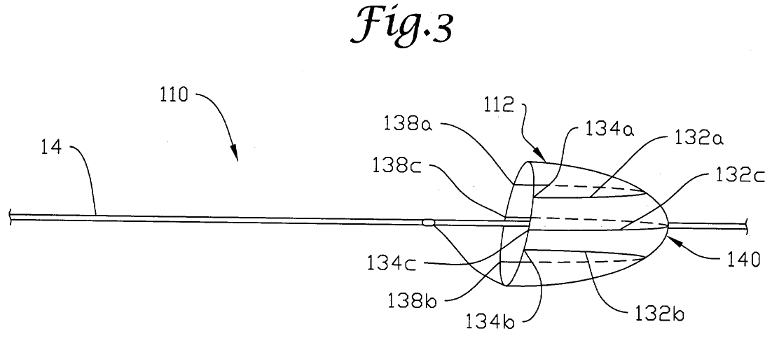

The number of support fibers 32a/b may also vary. For example, some embodiments of filtering device 10 include two as shown in FIG. 2. Alternatively, it may be appropriate to include one, or it may be appropriate to include more than two. For example, FIG. 3 illustrates another example filtering device 110 that includes filter 112 with three support fibers 132a/b/c.

Support fibers 132a/b/c of device 110 may be arranged in any appropriate manner. For example, fibers 132a/b may be configured essentially the same as fibers 32a/b in FIG. 2 and fiber 132c may extend across a distal apex 140 of filter 112. This configuration may be desirable for providing additional support adjacent apex 140, which may be the position of filter 112 that feels the brunt of the force associated with debris buildup.

Any number of the various ends of fibers 132a/b (as well as other fibers described herein) may be attached to frame 16. For example, both first ends 134a/b/c and second ends 138a/b/c may be attached to frame 16. However, some embodiments include any individual or combination of the aforementioned ends attached to frame 16. Additionally, the attachment point between fibers 132a/b/c and frame 16 (and/or membrane 18) may also vary along frame 16. In general, fibers 132a/b/c may be attached at any position along frame 16, in any configuration or arrangement with respect to one another (e.g., opposite one another, adjacent one another, randomly disposed, etc.), or with differing numbers of ends attached.

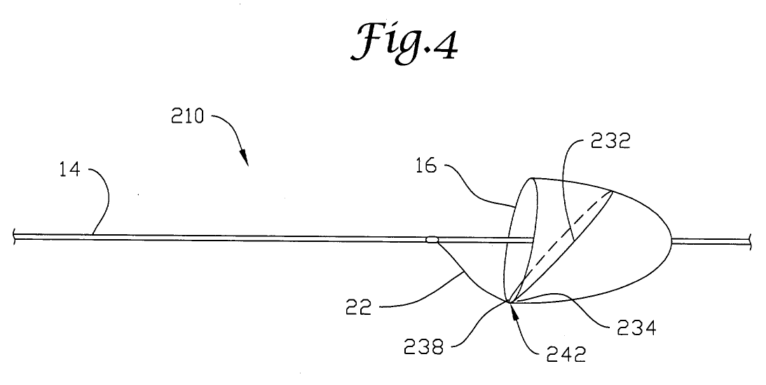

FIG. 4 is a side view of another example filtering device 210 where both first end 234 and second end 238 of support fiber 232 are attached to frame 16 adjacent the junction 242 of frame 16 and strut 22. This configuration may be appropriate for any of the devices described herein. The type of connection for this and any embodiment described herein may include using a connector similar to connector 24 (please see FIG. 1). Alternatively, other types of connection methods may be used including welding (e.g., resistance or laser welding), soldering, brazing, adhesive bonding, casting, molding including injection molding, mechanical bonding, thermal bonding, thermal forming, thermal-reforming (e.g., I/R heat flow or reflow), heat shrink techniques, and the like, or combinations thereof.

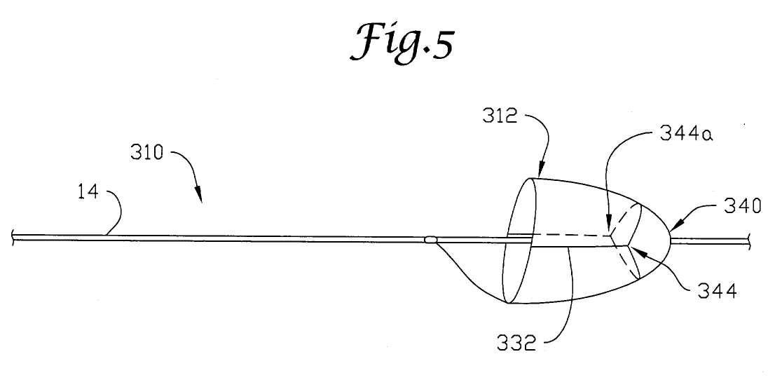

Another example filtering device 310 is shown in FIG. 5. Device 310 is essentially the same in form and function as any of the other devices described herein, except that support fiber 332 includes one or more bifurcation points 344. Bifurcation of fiber 332 may be desirable, for example, by increasing the area that fiber 332 can be spread over and provide support for filter membrane 18.

In some embodiments, bifurcation point 344 may be generally located adjacent distal apex 340 of filter 312. However, bifurcation point 344 can be disposed along any portion of fiber 332. The bifurcated portion of fiber 332 may or may not re-converge. For example, FIG. 5 shows fiber 332 spitting at bifurcation point 344 and then re-converging (at a position indicated by reference number 344a). This embodiment may be alternatively characterized as being the combination of two fibers, each having a bifurcation point, that merge or join into one fiber. In alternative embodiments, fiber 332 may include bifurcation point 344, which results in the defining of two fibers (each a portion of fiber 332) that may terminate at an ending point without reconverting.

Although the term bifurcation is understood to be the splitting of fiber 332 into two pathways, the invention is not intended to be limited to only the splitting into two pathways. Splitting into three (i.e., “trifurcation”) or more pathways is also within the scope of the invention. In embodiments where more than one bifurcation points 344 are included, the additional bifurcation points may be on separate fibers, serially located on one fiber, or both. It can be appreciated that these and other features of bifurcation and/or the inclusion of one or more bifurcation points 344 can be incorporated into any of the example embodiments described herein.

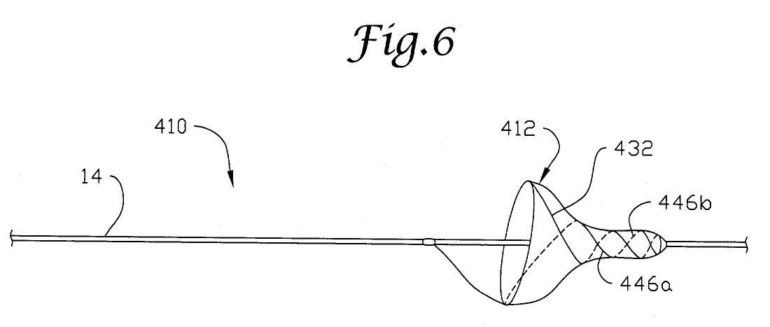

Another example filtering device 410 is shown in FIG. 6. Device 410 is essentially the same in form and function as any of the devices described herein, except that support fiber 432 is generally disposed about filter 412 in a helical or arcuate manner. In some embodiments, fiber 432 may include a first helical or arcuate region oriented in a first direction (indicated by reference number 446a) and a second helical or arcuate region oriented in a second direction (indicated by reference number 446b). The first and second directions may be opposite to one another, the same as one another, or be in essentially any appropriate relationship to one another.

FIG. 6 also illustrates that in some embodiments, the shape of filter 412 may also vary. For example, the distal portion of the filter may be narrowed as shown in FIG. 6. This feature may, for example, help provide structural support to the portion of filter 412. It can be appreciated, however, that essentially any appropriate shape can be used in conjunction with any of the filtering devices described herein.

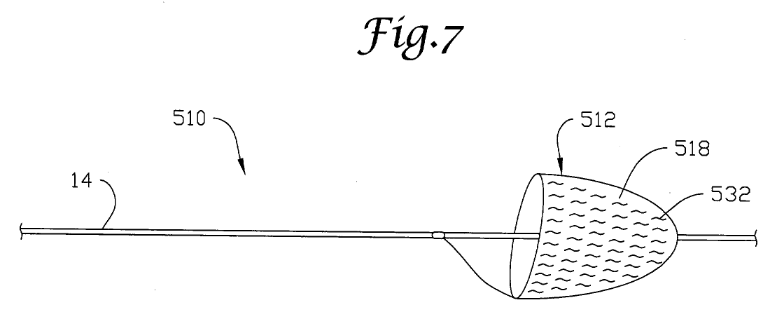

FIG. 7 is a side view of another example filtering device 510 that is essentially the same in form and function as any of the devices described herein except that filter 512 may include filter membrane 518 comprised of a reinforced or composite material. For example, filter membrane 518 may be comprised of a material reinforced by and/or embedded with fibers 532 that are dispersed throughout portions or all of filter membrane 518.

The materials suitable for filter membrane 518 and fibers 532 may vary. For example, filter membrane 518 may be comprised of a polymer and fibers 532 may be comprised of a generally stronger or more resilient polymer or metal. However, any appropriate material may be used for these structures including any of the materials disclosed herein. Additionally, the distribution of fibers 532 throughout filter membrane 518 may also vary. For example, the distribution of fibers 532 may be homogenous throughout filter membrane 518. Alternatively, fibers 532 may be distributed through only portions of filter membrane 518, be irregularly distributed, be more highly concentrated at particular positions (e.g., near the distal end of filter 512), etc.

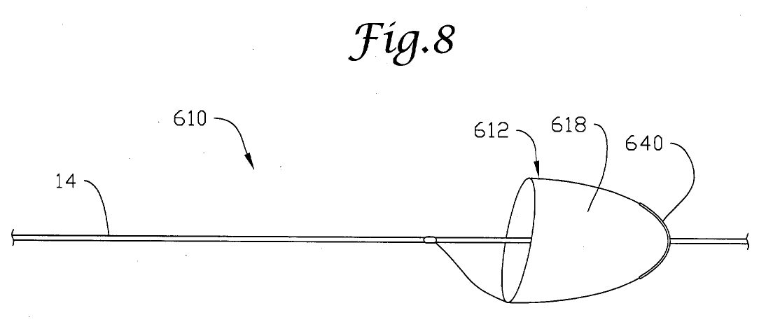

FIG. 8 is a side view of another example filtering device 610 that is essentially the same in form and function as any of the other device described herein except that only the distal apex region 640 of filter 612 is reinforced. Reinforcement of adjacent distal apex 640 may be accomplished in a number of ways. For example, distal apex region 640 may be thickened with additional layers of filter membrane 618. The additional layers of filter membrane 618 may be disposed along the inside surface of filter 612, the outside surface of filter 612, or both. Alternatively, distal apex region 640 may include one or more support fibers in a manner that is analogous to any of the embodiments described herein.

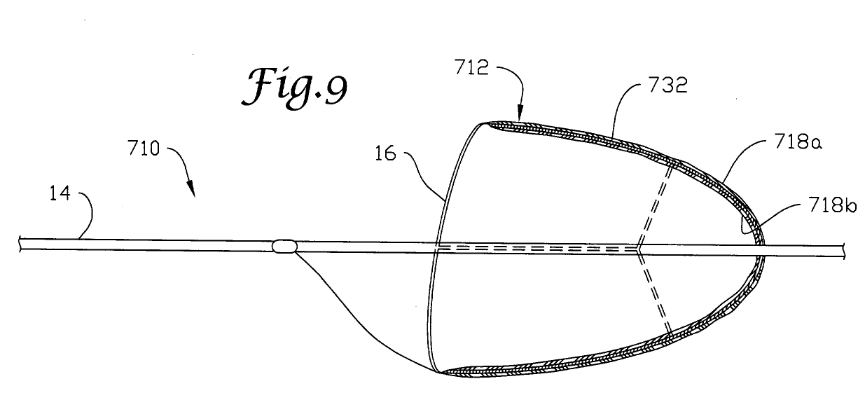

FIG. 9 is a partially cross-sectioned side view of another example filtering device 710 that is essentially the same in form and function as any of the other device described herein except that filter 712 is reinforced by disposing support fiber 732 between a plurality of filter membrane layers 718a/b. The form and composition of filter membrane layers 718a/b may be essentially the same any of the other embodiments described herein.

Additionally, the form and composition of fiber 732 may also be the same as any of the embodiments described herein. For example, fiber 732 may include bifurcations (as shown in phantom in FIG. 9) or other suitable multi-segment configurations or shapes, and/or may or may not include an end attached to frame 16. Alternatively, fiber 732 may be comprised of a reinforced fiber/membrane composite similar to that described above in relation to FIG. 7.

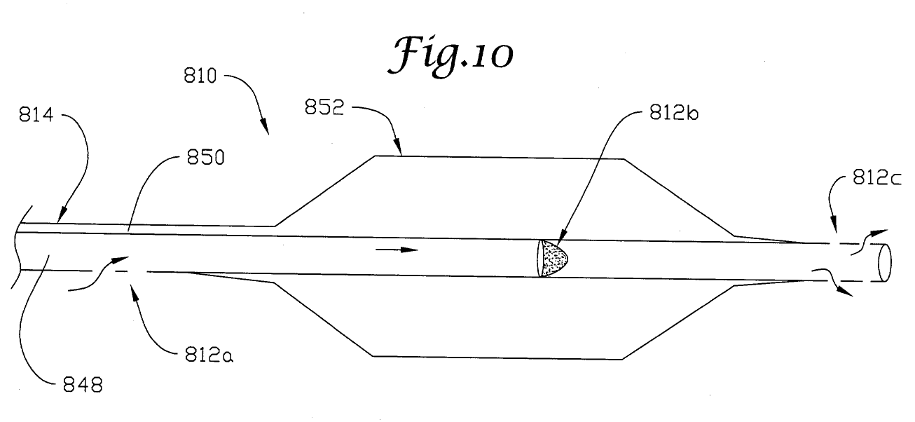

FIG. 10 is a side view of another example filtering device 810. Filtering device 810 may include one or more filters (indicated by reference numbers 812a, 812b, and 812c in FIG. 10) coupled to shaft 814. Shaft 814 may include a first lumen 848, a second lumen 850, and an inflatable member 852 coupled thereto. First lumen 848 may be a guidewire lumen and/or perfusion lumen for a medical device, for example a balloon catheter. Second lumen 850 may comprise an inflation lumen.

In at least some embodiments, filters 812a/b/c may be configured to filter fluid passing through at least one of the lumens, for example first lumen 848. For example, lumen 848 may be a perfusion lumen that allows blood to pass through when inflatable member 852 is inflated, which might otherwise occlude blood flow. Filters 812a and 812c may comprise one or more openings within shaft 814 that are configured to filter blood entering and/or exiting lumen 848. In some embodiments, the size (e.g., diameter) and/or arrangement of the openings may be configured so as to effectively filter debris.

Filter 812b may comprise a filter, which may be substantially similar to any of those described herein, disposed within lumen 848 so as to filter fluid passing through lumen 848. Filter 812b may be used independently from or in combination with one or both of filters 812a/c. In some embodiments, filter 812b may be hingedly disposed within lumen 848. This feature may allow filter 812b to be used within guidewire lumens of catheters and other medical devices. For example, filter 812b may configured for it to be “pivoted” upward against the wall surface of the tubular structure defining lumen 848. According to this embodiment, as a guidewire approaches and eventually contacts filter 812b, filter 812b pivots or swivels up to allow the guidewire to pass. When the guidewire is later retracted, filter 812b can swivel back down to the position appropriate for filtering debris.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An embolic protection filtering device, comprising:
an elongate shaft having a proximal end and a distal end;
a filter frame coupled to the shaft;
a filter membrane coupled to the filter frame; and
a membrane support fiber coupled to the filter membrane.

2. The filtering device of claim 1, further comprising one or more additional support fibers coupled to the filter membrane.

3. The filtering device of claim 1, wherein the filter has a distal apex and wherein the body region of the support fiber extends across the apex.

4. The filtering device of claim 3, further comprising one or more support fibers coupled to the filter membrane.

5. The filtering device of claim 1, wherein the support fiber includes a first end coupled to the filter frame.

6. The filtering device of claim 1, further comprising a filter body region including one or more bifurcations.

7. The filtering device of claim 6, wherein the one or more bifurcations are disposed adjacent a distal apex of the filter.

8. The filtering device of claim 6, wherein the bifurcated body region re-converges.

9. The filtering device of claim 8, wherein the support fiber includes a first end coupled to the filter frame.

10. The filtering device of claim 1, wherein the body region includes a arcuate region that is substantially arcuate is shape.

11. The filtering device of claim 1, wherein the filter frame includes a strut.

12. The filtering device of claim 11, wherein the support fiber includes a first end coupled to the filter frame.

13. The filtering device of claim 12, wherein the support fiber includes a second end coupled to the filter frame.

14. The filtering device of claim 1, further comprising a filter body region includes a first helical region that is substantially helical in shape and configured in a first helical direction.

15. The filtering device of claim 14, wherein the body region includes a second helical region that is substantially helical in shape and configured in a second helical direction, the second helical direction being generally opposite of the first helical direction.

16. The filtering device of claim 15, wherein the support fiber includes a first end coupled to the filter frame.

17. The filtering device of claim 1, further comprising a second filter membrane layer and wherein the support fiber, is disposed between the filter membrane and the second filter membrane layer.

18. An embolic protection filtering device, comprising:
an elongate shaft having a proximal end and a distal end;
a filter coupled to the shaft, the filter including a filter frame, a filter material coupled to the filter frame, and one or more struts extending between the frame and the shaft;
reinforcing for the filter material.

19. The filtering device of claim 18, wherein the reinforcing of the filter material includes a plurality of fibers embedded within the filter material.

20. The filtering device of claim 18, wherein the reinforcing of the filter material includes a distal region of the filter that is thickened by additional filter material.

21. The filtering device of claim 18, wherein the reinforcing of the filter material includes a support fiber having a first end coupled to the filter frame and a body region disposed adjacent the filter material.

22. An embolic protection filtering device, comprising:
an elongate shaft having a proximal end and a distal end;
a filter frame coupled to the shaft, the filter frame including a filter loop and one or more struts extending between the loop and the shaft;
a filter membrane coupled to the filter frame adjacent the filter loop and extending distally therefrom to define a filter; and
wherein the filter material is comprised of a composite of a first material and a second reinforcing material embedded within the first material.

23. An embolic protection filtering device, comprising:
an elongate shaft having a proximal end and a distal end;
a filter frame coupled to the shaft, the filter frame including a filter loop and one or more struts extending between the loop and the shaft;
a filter membrane coupled to the filter frame adjacent the filter loop and extending distally therefrom to define a filter; and
wherein the filter material substantially thickened adjacent a distal end of the filter so as to provide structural support to the filter.

24. An intravascular balloon catheter, comprising:
an elongate tubular shaft having a proximal end, a distal end, and an inflation lumen extending therethrough;
an expandable balloon coupled to the shaft adjacent the distal end, the balloon being in fluid communication with the inflation balloon and including a proximal end and a distal end;
wherein shaft includes perfusion member including a proximal perfusion orifice, a distal perfusion orifice, and a perfusion lumen extending therebetween;
a filtering member coupled to the perfusion member.

25. The balloon catheter of claim 24, wherein the filtering member comprises a plurality of openings disposed at the proximal perfusion orifice.

26. The balloon catheter of claim 24, wherein the filtering member comprises a plurality of openings disposed at the distal perfusion orifice.

27. The balloon catheter of claim 24, wherein the filtering member comprises a filter disposed within the perfusion lumen.

28. An embolic protection filtering device, comprising:
an elongate shaft;
a filter frame coupled to the shaft, the filter frame including a filter mouth and one or more struts extending between the filter mouth and the shaft;
a filter material coupled to the filter frame adjacent the filter mouth and extending distally therefrom to define a filter; and
a membrane support fiber coupled to the filter membrane, the fiber including a first end coupled to the filter mouth, a body region disposed adjacent the filter membrane, and a second end coupled to the filter mouth.

29. The filtering device of claim 28, further comprising one or more additional support fibers coupled to the filter membrane.

30. The filtering device of claim 28, wherein the filter has a distal apex and wherein the body region of the support fiber extends across the apex.

31. The filtering device of claim 30, further comprising one or more support fibers coupled to the filter membrane.

32. The filtering device of claim 28, wherein the body region includes one or more bifurcations.

33. The filtering device of claim 32, wherein the one or more bifurcations are disposed adjacent a distal apex of the filter.

34. The filtering device of claim 33, wherein the bifurcated body region re-converges.

35. The filtering device of claim 28, wherein the body region includes a arcuate region that is substantially arcuate is shape.

36. The filtering device of claim 28, wherein the strut and the filter mouth are attached to one another at an attachment point, and wherein the first end of the support fiber is coupled to the filter loop adjacent the attachment point.

37. The filtering device of claim 36, wherein the second end is coupled to the filter mouth adjacent the attachment point.

38. The filtering device of claim 28, wherein the body region includes a first helical region that is substantially helical in shape and configured in a first helical direction.

39. The filtering device of claim 38, wherein the body region includes a second helical region that is substantially helical in shape and configured in a second helical direction, the second helical direction being generally opposite of the first helical direction.

40. The filtering device of claim 28, further comprising a second filter membrane layer and wherein the support fiber is disposed between the filter membrane and the second filter membrane layer.
